(19) United States
(12) Patent Application Publication
(10) Pub. No.: US 2005/0026897 A1
(43) Pub. Date: Feb. 3, 2005

(54) Trinuclear copper-based compound and ligand for nucleic acid scission and anticancer treatment

(76) Inventors: Steven E. Rokita, Silver Spring, MD (US); Kenneth D Karlin, Owings Mills, MD (US); Kristi J Humphreys, Oakland, CA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/475,860

(22) PCT Filed: May 20, 2002
(86) PCT No.: PCT/US02/15136

Related U.S. Application Data
(62) Division of application No. 60/292,202, filed on May 18, 2001.

(51) Int. Cl. A61K 31/655; A61K 31/555

(52) U.S. Cl. 514/185; 514/151

(57) ABSTRACT

The present invention is related to a novel method for splitting nucleic acids at specific points on a complementary nucleic acid segment using a trinuclear copper-based compound of formula (I). Additionally, the present invention is related to a novel treatment of cancer, tumors, and cancer cells using a trinuclear copper-based compound of formula (I) or a naked ligand of formula (II).

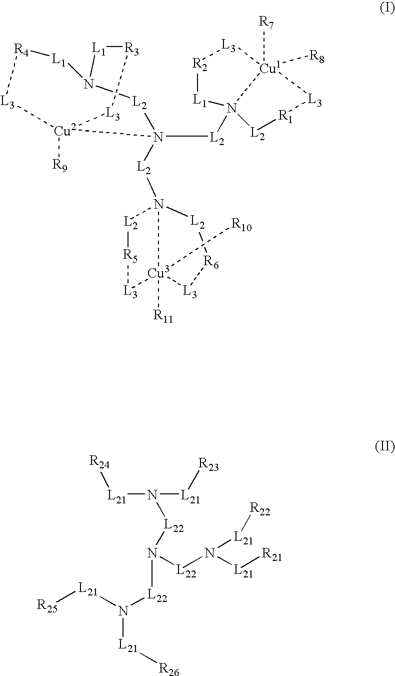

This application claims the benefit of U.S. Provisional Application No. 60/292,204, filed May 18, 2001.

The United States Government has rights in this invention pursuant to Grants No. GM47531 and GM28962 awarded by the National Institute of Health (NIH).

### FIELD OF INVENTION

The present invention is related to a novel method for splitting nucleic acids at specific points on a complementary nucleic acid segment using a trinuclear copper-based compound. Additionally, the present invention is related to a novel treatment of cancer, tumors, and cancer cells using a trinuclear copper-based compound or the naked ligand.

### BACKGROUND OF INVENTION

A number of transition metal complexes have been found to be able to differentiate between double vs. single-stranded DNA or B vs. Z helical forms of DNA through noncovalent recognition. This selectivity is primarily due to the binding of the transition metal complex in either the major or minor groove of duplex structures or in association with the nucleobases in unpaired strands. The electron-rich character of the nucleobases often makes them strong ligands for metals and efficient targets of oxidation. Guanine has been shown to have the highest affinity for coordination to transition metal ions and it is also the most easily oxidized, followed by adenine, cytosine and thymine (in order of ease of oxidation). Although base oxidation can be highly specific and directed to one site, strand scission has been shown to result from base oxidation only after treatment with subsequent heat and alkaline conditions.

However, direct strand scission does not necessarily require any special treatment to detect the sites of reaction. Some complexes that exhibit direct strand cleavage in conjunction with sequence specificity are bleomycin.Fe(II) and the metallointercalator, [Rh(phen)₂phi]³⁺. Although there is both a structural and a sequence requirement in each of these cases, the recognition criteria are not sufficiently unique to limit the number of target sites in DNA. Scission may be targeted specifically to one site by incorporating known DNA recognition elements into the ligand suprastructure of a well-characterized nucleolytic agent such as EDTA.Fe(II), which, when underivatized, promotes oxidative cleavage of DNA in a random fashion without nucleotide sequence selectivity. While this approach localizes cleavage to a site where the recognition element binds to DNA, the reaction is rarely constrained to a single nucleotide. Strand cleavage frequently extends over more than 5 bases. A longstanding goal of considerable interest has been to construct transition metal complexes that can mediate direct and specific strand scission targeted to a single base with a significantly high level of recognition such that cleavage occurs at a limited number of sites along a target polynucleotide.

Most investigations focusing on oxidative strand scission of DNA by transition metals have typically relied on mononuclear complexes. Among these complexes, bis(1,10-phenanthroline)copper, [Cu(OP)₂]²⁺, has been studied extensively due to its high nucleolytic efficiency. The cleavage pattern induced by [Cu(OP)₂]²⁺ is predominantly sequence-neutral, although some variability in intensity due to local perturbations of DNA structure affects its efficiency. Also a slight, but distinct, preference for cleavage at 5′-AT-3′ and 5′-GT-3′ sites has been observed. Otherwise, [Cu(OP)₂]²⁺ like EDTA.Fe(II) may be conjugated to binding elements such as proteins and complementary sequences of RNA or DNA that possess affinity for specific sites on DNA. Still, multiple sites adjacent to the locus of recognition are typically oxidized by these complexes even when tethered to a DNA recognition element.

An example of research into oxidative strand scission of DNA by transition metals is detailed in the article entitled “A new trinuclear complex and its reactions with plasmid DNA” by Steven T. Frey, Helen H. J. Sun, Narasimha N. Murthy, and Kenneth D. Karlin, Inorganica Chimica Acta 242 (1996) 329-338 (hereinafter referred to as “Frey”). This article discusses the synthesis of a novel trinuclear copper(II) complex and its reactivity with plasmid pBR322. While Frey discloses a trinuclear compound within the disclosure of formulas I and II of the present invention, it is noted that Frey does not disclose that these compounds possess the surprising ability to treat cancer or to split nucleic acids at specific locations adjacent to a complementary nucleic acid segment. In fact, on page 335 of the article, Frey teaches away from the splitting of nucleic acids at a specific position by noting “that cleavage [with the trinuclear compound] is not sequence specific.” Therefore, Frey is not relevant to the novelty or nonobviousness of the present invention.

### SUMMARY OF INVENTION

The present invention is based on the discovery that certain trinuclear copper-based compounds possess the ability to recognize and promote scission of a nucleic acid at specific positions. Additionally, it has been discovered that the trinuclear copper-based compounds and the naked ligand possess the ability to treat cancer.

Thus, the invention is directed towards a method of treating cancer in a patient in need thereof, comprising administering to a patient a cancer-treating effective amount of a compound of formula I, 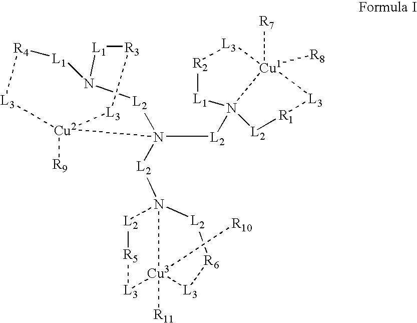
wherein
R₁-R₆ are each independently a 5 to 6 membered heterocycle containing 1-3 nitrogen atoms and optionally one oxygen atom, with the remainder of the atoms being carbon atoms, wherein the heterocycle is linked to a respective linkage L₃ through a nitrogen atom of the heterocycle, and wherein the heterocycle is linked to a respective linkage L₁ through any of the nitrogen or carbon atoms of the heterocycle other than the nitrogen atom that links to linkage L₃; and wherein the 5 to 6 membered heterocycle is unsubstituted or substituted with 1-3 substituents selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide;
R₇-R₁₁ are each independently an anion or uncharged species;
each L₁ is independently an ethyl, methyl, or ether linkage;
each L₃ is a direct bond; and
each L₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide, and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

The invention is further directed towards a method of treating a cancer tumor, said method comprising administering to the cancer tumor a cancer tumor-treating effective amount of a compound of formula I.

The invention is additionally directed towards a method of treating cancer cells, said method comprising administering to the cancer cells a cancer cell-treating effective amount of a compound of formula I.

The invention is further directed towards a use of a compound of formula I to treat cancer.

The invention is further directed towards the use of a compound of formula I to prepare a medicament suitable for treating cancer.

The invention is further directed towards a method of splitting a nucleic acid segment at a specific position thereon, wherein said method comprises (a) providing a first nucleic acid segment having (i) an n position, wherein said n position is occupied by a first purine nucleotide that is non-complementary to a corresponding position x on a second nucleic acid segment, and (ii) an n+1 position which is occupied by a guanine residue, wherein said n+1 position is located directly adjacent to the n position and upstream towards the 5′ end of the first nucleic acid segment, and a second nucleic acid segment which is complementary to the first nucleic acid segment upstream from the position x, wherein the second nucleic acid segment is located either on a different or the same nucleic acid strand as the first nucleic acid segment; and (b) contacting at least the second nucleic acid segment with a compound of formula I for a time sufficient to split the nucleic acid at the position x of the second nucleic acid segment.

The invention is further directed towards the use of a compound of formula I to split a nucleic acid segment at a specific position x thereon, wherein the specific position x is located on a second nucleic acid segment which is complementary to a first nucleic acid segment upstream from the position x, wherein the position x is non-complementary to an n position on the first nucleic acid segment, wherein the n position is occupied by a first purine nucleotide, and the n position is directly adjacent to an n+1 position and is located upstream towards the 5′ end of the first nucleic acid segment, wherein said n+1 position is occupied by a guanine.

The invention is further directed towards a method of treating cancer in a patient in need thereof, said method comprising administering to a patient a cancer-treating effective amount of a compound of formula II, 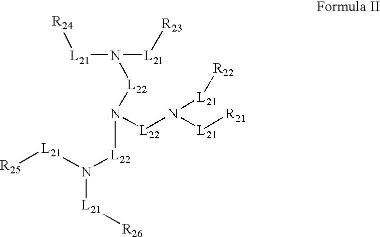
wherein
R₂₁-R₂₆ are each independently a 5 to 6 membered heterocycle containing 1-3 nitrogen atoms and optionally one oxygen atom, with the remainder of the atoms being carbon atoms, wherein the heterocycle is linked to a respective linker L₂₁through a carbon or nitrogen atom of the heterocycle; and wherein the 5 to 6 membered heterocycle is unsubstituted or substituted with halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, phenyloxycarbonylamino, naphthyloxycarbonylamino, semicarbazido, heteroaryl, 4-acetoxyphenyloxy, phenyl or acetamide;
each L₂₁ is independently an ethyl, methyl, or ether linkage; and
each L₂₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide, and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

The invention is further directed towards a method of treating a cancer tumor, said method comprising administering to the cancer tumor a cancer tumor-treating effective amount of a compound of formula II.

The invention is further directed towards a method of treating cancer cells, said method comprising administering to the cancer cells a cancer cell-treating effective amount of a compound of formula II.

The invention is further directed towards a use of a compound of formula II to treat cancer.

The invention is further directed towards the use of a compound of formula II to prepare a medicament suitable for treating cancer.

The invention is also directed towards a pharmaceutical composition containing a pharmaceutically effective amount of at least one compound of formula I.

The invention is also directed towards a pharmaceutical composition containing a pharmaceutically effective amount of at least one compound of formula II.

### BRIEF DESCRIPTION OF THE DRAWINGS

FIG. 1 is a depiction of the secondary structure of single-stranded nucleic acid segment OD1 having a hairpin arrangement. Pairing of the hairpin is designated by the (—). The primary cleavage site is indicated by a “x” and secondary sites by a small arrowhead.

FIG. 2 is a depiction of the secondary structure of double stranded nucleic acid segments OD3+OD4. The primary cleavage site is indicated by an “x.”

FIG. 3 is an autoradiogram of a 20% polyacrylamide denaturing gel (7 M urea) showing cleavage products of 100 nM 5′-³²P-labeled OD1 incubated with a compound of formula I and MPA (3-mercaptopropionic acid) for 15 minutes in sodium phosphate (10 mM, pH 7.5) at ambient temperature. Lane 1 depicts OD1 alone. Lane 2 depicts OD1 with 5 μM formula I. Lane 3 depicts OD1 with 5 mM MPA. Lane 4-6 depict 0.5, 1, and 5 μM formula I with 5 mM MPA and OD1. Lane 7 depicts OD1+OD2 with 10 μM formula 1 and 5 mM MPA. Lane 8 depicts A+G sequencing lane.

### DETAILED DESCRIPTION OF THE PREFERRED EMBODIMENTS

The present invention is directed to novel uses of trinuclear copper-based compounds and the naked ligand thereof.

The term “complementary” is intended to define the relationship between the pairing of purines and pyrimidines in a nucleic acid. For example, one complementary pairing is the pairing of adenine with thymine or uracil. Another complementary pairing is the pairing of guanine with cytosine.

The term “purine” includes guanine and adenine.

The term “pyrimidine” includes thymine, uracil and cytosine.

The term “nucleic acid” includes DNA and RNA.

The term “segment” is intended to define a region of a nucleic acid sequence on a nucleic acid strand.

The term “n position” is intended to define a position on a first nucleic acid segment that is occupied by the first purine nucleotide that is non-complementary to the corresponding position on a second nucleic acid segment, regardless of whether the second nucleic acid segment is contained on the same nucleic acid strand or a different nucleic acid strand as the first nucleic acid segment containing the n position.

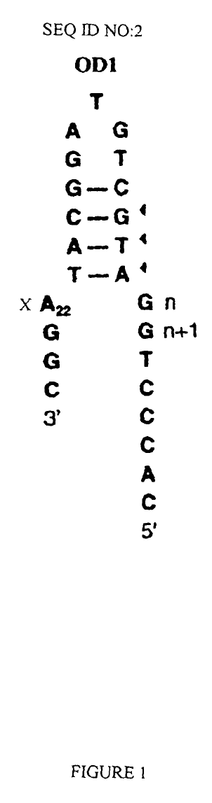

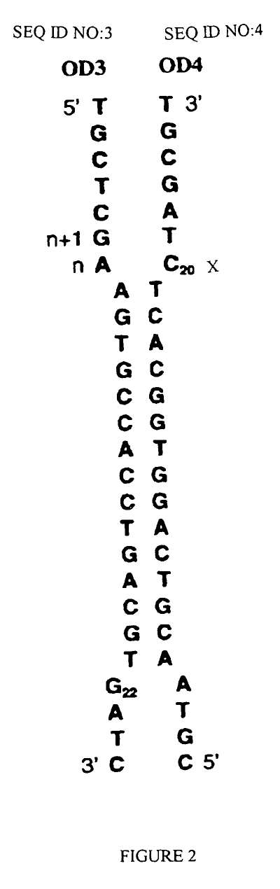

The term “n+1 position” is intended to define a position on a first nucleic acid segment that is located directly adjacent to the n position on the first nucleic acid segment. The n+1 position is located directly upstream from the n position and towards the 5′ end of the nucleic acid (see FIGS. 1 and 2).

The term “position x” is intended to define a position on a second nucleic acid segment that directly corresponds to (i.e., is across from) the n position on the first nucleic acid segment.

Complete complementarity between the second nucleic acid segment and the first nucleic acid segment (with the exception of the n and x positions) is not required. It is preferred that the second nucleic acid segment be complementary to the first nucleic acid segment upstream from the position x. This complementarity can be, for example, at least 3 nucleotides, preferably at least 4 nucleotides (as shown in FIG. 1), and more preferably at least 5 nucleotides. FIG. 2 shows a complementarity of 15 nucleotides. As shown in FIGS. 1 and 2, the second nucleic acid segment may be located on a different nucleic acid or on the same nucleic acid as the first nucleic acid segment.

In the compound of formula I, the groups labeled R₁-R₆ are each independently a 5 to 6 membered heterocycle containing 1-3 nitrogen atoms and optionally one oxygen atom, with the remainder of the atoms being carbon atoms. These heterocycles include, but are not limited to, pyrrolyl, 2-H pyrrolyl, 3H-pyrrolyl, pyrazolyl, 2H-imidazolyl, 1, 2, 3-triazolyl, 1, 2, 4-triazolyl, isoxozolyl, oxazolyl, 1, 2, 3-oxadiazolyl, 1, 2, 4-oxadiazolyl, 1, 2, 5-oxadiazolyl, 1, 3, 4-oxadiazolyl, 1, 2, 3, 4-oxatriazolyl, 1, 2, 3, 5-oxatriazolyl, pyridyl, pyridazyl, pyrimidinyl, pyrazyl, piperazyl, 1, 3, 5-triazyl, 1, 2, 4-triazyl, 1, 2, 3-triazyl, 4H-1, 2-oxazyl, 2H-1, 3-oxazyl, 6H-1, 3-oxazyl, 6H-1, 2-oxazyl, 1, 4-oxazyl, 2H-1, 2-oxazyl, 4H-1, 4-oxazyl, 1, 2, 4-oxadiazyl, 1, 3, 5-oxadiazyl, pyrrolidinyl, piperidinyl, piperazinyl, pyrazinyl, 1, 2, 3, 6-tetrahydropyridinyl, pyrazolinyl, pyrazolidinyl, pyridinyl, pyrazolyl, triazolyl, pyrazinyl, oxazolyl, pyridazinal, triazinyl, and morpholyl.

The 5 to 6 membered heterocycle is either unsubstituted or substituted with 1 to 3 substituents selected from halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide.

In formula I, groups R₇-R₁₁ are each independently an anion or uncharged species. Any physiologically acceptable or pharmaceutically acceptable anion can be used as a substituent for R₇-R₁₁. Said acceptable anions include, but are not limited to, any thiolate, nitrate, chloride, acetate, perchlorate, phosphate, bromide, fluoride, iodide, sulfate, trifluoromethanesulfonate, hexafluorophosphate, hexafluoroantimonate

or any halide anion.

Additionally, in formula I, each L₁ is independently an ethyl, methyl, or ether linkage, and L₃ is a direct bond. It is also preferred that the L₁ linkage is linked to a carbon atom of the heterocycle. Each L₂ is independently (a) a C₁-C₆ alkyl which may optionally be interrupted with one or more ether linkages, wherein the C₁-C₆ alkyl is unsubstituted or substituted with 1 to 3 substituents each of which is independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

All references to “halogen” include fluorine, chlorine, bromine, and iodine. All references to the alkyl groups include branched or unbranched alkyl groups. All references to “heteroaryl” are intended to include a monocyclic aromatic hydrocarbon group having 5 or 6 ring atoms, or a bicyclic aromatic group having 8 to 10 atoms, each containing 1-3 heteroatoms selected from O, S or N, with the remainder of the atoms being carbon. Examples of suitable heteroaryls include pyridinyl, pyrimidinyl, and pyrazinyl, pyridazinyl, pyrrolyl, furanyl, oxazolyl, isoxazolyl, thiazolyl, isobenzofuryl, benzofuryl, benzo[b]thiophenyl, benzo[c]thiophenyl, indolyl, 3H-indolyl, 1H-indolyl, cyclopenta[b]pyridinyl, pyrano[3,4-b]pyrrolyl, indazolyl, benzisoxazolyl, benzoxazolyl, 2,1-benzisoxazolyl, 2H-1-benzopyranyl, 2H-1-benzoyran-2-yl, 4H-1-benzopyran-4-yl, 1H-2-benzopyran-1-yl, 3H-2-benzopyran-1-yl, quinolinyl, isoquinolinyl, cinnolinyl, quinazolinyl, 1,8-napthtyridinyl, 1,7-napthtyridinyl, 1,5-napthtyridinyl, 1,6-napthtyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl, 4H-1,4-benzoxazinyl, and the like.

In the compound of formula II, groups R₂₁-R₂₆ are each independently a 5 to 6 membered heterocycle containing 1-3 nitrogen atoms and optionally one oxygen atom, with the remainder of the atoms being carbon atoms. These heterocycles include, but are not limited to, pyrrolyl, 2-H pyrrolyl, 3H-pyrrolyl, pyrazolyl, 2H-imidazolyl, 1, 2, 3-triazolyl, 1, 2, 4-triazolyl, isoxozolyl, oxazolyl, 1, 2, 3-oxadiazolyl, 1, 2, 4-oxadiazolyl, 1, 2, 5-oxadiazolyl, 1, 3, 4-oxadiazolyl, 1, 2, 3, 4-oxatriazolyl, 1, 2, 3, 5-oxatriazolyl, pyridyl, pyridazyl, pyrimidinyl, pyrazyl, piperazyl, 1, 3, 5-triazyl, 1, 2, 4-triazyl, 1, 2, 3-triazyl, 4H-1, 2-oxazyl, 2H-1, 3-oxazyl, 6H-1, 3-oxazyl, 6H-1, 2-oxazyl, 1, 4-oxazyl, 2H-1, 2-oxazyl, 4H-1, 4-oxazyl, 1, 2, 4-oxadiazyl, 1, 3, 5-oxadiazyl, pyrrolidinyl, piperidinyl, piperazinyl, pyrazinyl, 1, 2, 3, 6-tetrahydropyridinyl, pyrazolinyl, pyrazolidinyl, pyridinyl, pyrazolyl, triazolyl, pyrazinyl, oxazolyl, pyridazinal, triazinyl, and morpholyl.

The 5 to 6 membered heterocycle is either unsubstituted or substituted with 1 to 3 substituents selected from halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide.

Additionally, in formula II, each L₂₁ linkage is independently an ethyl, methyl, or ether linkage. Preferably, the L₂₁ linkage is linked to a carbon atom of the heterocycle. Each L₂₂ is independently (a) a C₁-C₆ alkyl which may optionally be interrupted with one or more ether linkages, wherein the C₁-C₆ alkyl is unsubstituted or substituted with 1 to 3 substituents each of which is independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

The compounds of formula I have been found to be effective in treating cancer. Some types of cancer that the compounds of formulas I and II have been found to be effective in treating are leukemia, non-small cell lung cancer, colon cancer, central nervous system cancer, melanoma, ovarian cancer, renal cancer, ovarian cancer, cancer of the head and neck, bladder cancer, small cell cancer of the lung, squamous-cell carcinomas of the head, neck, esophagus, skin, and the genitourinary tract, including the cervix, vulva, scrotum, and penis, prostate cancer, and breast cancer. The compounds of formulas I and II are, therefore, suitable for use in methods for treating cancer, cancer cells, or tumors, whether the compound of formula I or II is used alone or in conjunction with another compound of formula I or formula II or another known anti-cancer agent.

Such methods comprise administering to a patient in need of such treatment an anti-cancer, anti-tumor, or anti-cancer cell effective amount (hereinafter “effective amount”) of one or more compounds of formulas I and/or II. The effective amount of the compound(s) of formulas I and/or II are preferably administered in any conventional form suitable for oral administration, for example in the form of a tablet, caplet, capsule, beadlet or powder. Additionally, dosage forms include troches, dispersions, suspensions, solutions, injections, infusions, creams, ointments, aerosols, and the like. These administration forms may be presented in unit dosage form and may be prepared by any of the methods well-known in the art of pharmacy. The compound(s) of formula I and/or II is/are present in an amount of from 1 to 99% by weight, based upon the total weight of the dosage form, for example from 10 to 50% by weight.

Additionally, the compound(s) of formula I and/or II can be administered in any other form suitable for rectal, topical, parenteral, intraperitoneal, ocular, pulmonary, inhalation, intramuscular, intravenous, and vaginal administration. The compound(s) of formula I and/or formula II is/are present in the dosage form in an amount of from 1 to 99% by weight, based upon the total weight of the dosage form, for example from 10 to 50% by weight.

The magnitude of a dose administered, however, varies according to the age, weight, sex, and response of the individual patient. In general, the daily dose range of a compound of formula I or II (or of any mixture thereof) is within the range of from about 0.001 mg to about 100 mg per kg body weight of a mammal, preferably 0.01 mg to about 10 mg per kg, and most preferably 0.1 to 1 mg per kg, in single or divided doses. On the other hand, it may be necessary to use dosages outside these limits in some cases. These dosages may fall within the range of 0.00001 to 500 mg administered to the patient per day. When the composition is in the form of an oral composition, the weight of the compound(s) of formula I and/or formula II in the composition may be in the range of from 0.00001 to 500 mg, such as from 5 to 250 mg or from 10 to 200 mg. If the compound(s) of formula I and/or formula II is/are in the form of a tablet, the tablet may be uncoated or coated and the coating may be a conventional coating and the coating may be applied by a conventional method.

A pharmaceutical composition with a compound (or compounds) of formula I and/or formula II as an active ingredient (or a pharmaceutically acceptable salt thereof), may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids. Said pharmaceutical composition contains a pharmaceutically effective amount of at least one compound of formula I and/or formula II. Further, the pharmaceutical composition is pharmaceutically effective against cancer, including leukemia, non-small cell lung cancer, colon cancer, central nervous system cancer, melanoma, ovarian cancer, renal cancer, ovarian cancer, cancer of the head and neck, bladder cancer, small cell cancer of the lung, squamous-cell carcinomas of the head, neck, esophagus, skin, and the genitourinary tract, including the cervix, vulva, scrotum, and penis, prostate cancer, and breast cancer.

The dosage may be administered in either one single dosage, two dosages, or in more than two dosages per day.

The compounds of formula I and formula II have been found to exhibit a remarkable ability to promote specific strand scission at junctions between single- and double-stranded DNA. Strand scission occurs at the position x at the junction of a hairpin or frayed duplex structure and is not dependent on the identity of the base at which cleavage occurs.

Scission minimally requires a purine at the first unpaired position (the n position) and a guanine at the n+1 position on the first nucleic acid segment. Selective strand scission is preferably conducted in the presence of dioxygen.

The time required to split the nucleic acid is normally between 0 (instant) and 60 minutes. However, the time required to split the nucleic acid may be adjusted according to the composition of the nucleic acid segments and the presence and amount of dioxygen. Further the compound(s) of formula I can be applied to the second nucleic acid segment alone, the first nucleic acid alone, or both the first and second nucleic acid segments either simultaneously or separately. The first and second nucleic acid segments are preferably longer than 5 nucleotides in length, more preferably between 5 and 100,000 nucleotides in length, even more preferably between 5 and 50,000 nucleotides in length, even further preferred is a segment that is between 5 and 10,000 nucleotides in length, and most preferred is a segment that is between 5 and 1,000 nucleotides in length. Also preferred are segments of between 10, 20, or 30 and 1,000 nucleotides in length.

The following are incorporated by reference in their entirety:
1) “Recognition and Strand Scission at Junctions between Single- and Double-stranded DNA by a Trinuclear Copper Complex” by Kristi J. Humphries, Kenneth D. Karlin, and Steven E. Rokita, J. Am. Chem Soc. 2001, 123, 5588-5589.
2) The Handbook of Chemistry and Physics (82nd Edition) edited by David R. Lide.
3) “A new trinuclear complex and its reactions with plasmid DNA” by Steven T. Frey, Helen H. J. Sun, Narasimha N. Murthy, and Kenneth D. Karlin, Inorganica Chimica Acta 242 (1996) 329-338.
4) “Targeted Strand Scission of DNA Substrates by a Tricopper II Coordination Complex” by Kristi J. Humphries, Kenneth D. Karlin, and Steven E. Rokita, J. Am. Chem Soc. 2002, 124, (in press).
5) “Goodman and Gilman's The Pharmaceutical Basis of Therapeutics” edited by Alfred Goodman Gilman, Theodore W. Rall, Alan S. Nies, and Palmer Taylor (8th Edition).
6) “Goodman and Gilman's The Pharmaceutical Basis of Therapeutics” edited by Joel G. Hardman, Lee E. Limbard, Perry B. Molinoff, Raymond W. Ruddon (9th Edition).
7) “Goodman and Gilman's The Pharmaceutical Basis of Therapeutics” edited by Joel G. Hardman, Lee E. Limbard, and Alfred Goodman Gilman (10th Edition).

### EXAMPLES

### Example 1

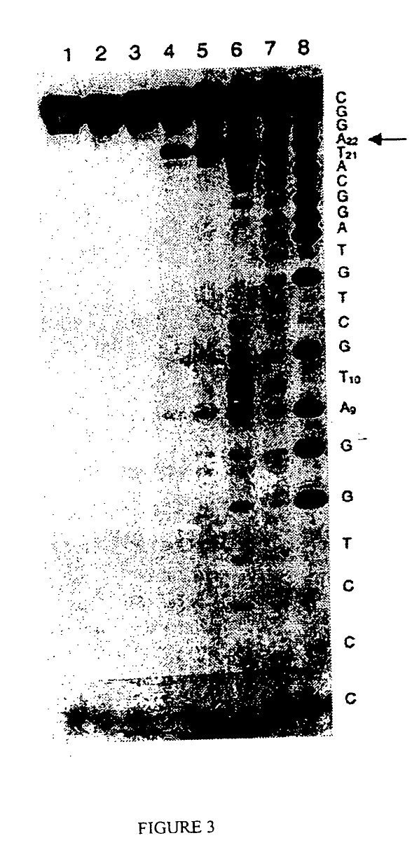

A hairpin forming oligonucleotide (OD1) (SEQ ID NO. 2) (see FIG. 1) was treated with a compound of formula I in the presence of excess MPA and it was then quenched with dithiocarbamic acid. PAGE (polyacrylamid gel electrophoresis) analysis revealed direct and specific strange scission at position A₂₂ (see FIG. 3, lanes 4 and 5). Quantitation of the products by phosphorimage analysis indicated that 78% of the observed cleavage occurred at A₂₂ in the presence of 0.5 μM of a compound of formula I and 5 mM MPA. Selectivity was reduced to 66% if the concentration of the compound of formula I is increased 10-fold to 5 μm, due in part to an increase in reactivity primarily at A₉, T₁₀ and T₂₁ (FIG. 3, lane 6). In the absence of either the thiol or the compound of formula I, no cleavage of OD1 was observed (FIG. 3, lanes 2 and 3). In addition, there was no enhancement of reaction at A₉, T₁₀ or T₂₁. Instead, strand scission proceeded with equivalent efficiency at all sites, much like results with Cu(OP)₂²⁺ and OD1+OD2, wherein OD2 is 5′ TATGCCTACAGCATCCAGGGTG 3′ (SEQ ID. NO 1). The persistent specificity for A₂₂ when OD2 is present was likely due to residual hairpin formed in competition with duplex DNA. Also, the unique modification at A₂₂ appears to be dominated by the recognition properties of the compound of formula I rather than the intrinsic reactivity of DNA since A₂₂ is not a major target of reaction with Cu(OP)₂²⁺ and either the hairpin-forming OD1 or the duplex-forming OD1+OD2.

### Example 2

When the reaction products of OD1 and a compound of formula I were treated with piperidine, a 40% enhancement in strand scission resulted, but the relative selectivity remained unchanged. Lack of reaction at other adenines indicated that the complex did not recognize a specific base, but instead targeted a distinct structural feature of the deoxyoligonucleotide. This observation is supported by having compared the specificity of scission to the hairpin conformation of OD1, a structure that was independently confirmed via chemical modification using CoCl₂ and KBr with oxone and KMnO₄. Position A₂₂ is directly 3′ to the stem region which contains A₉ and T₁₀ (see FIG. 1). The proximity of these target residues suggested a common site for association of a compound of formula I. A reaction comparable to that at A₂₂ was not observed in the single-stranded loop region of the hairpin, and therefore this structure does not contain the necessary recognition elements for strand scission.

### Example 3

To determine the origin of specificity for the trinuclear copper complex of formula I, a new target (OD3+OD4) was designed to present a central duplex region flanked by 3′ and 5′ single-stranded regions on both strands (FIG. 2). This provided two junctions between single- and double-stranded DNA in which a cytosine (C₂₀) and a guanine (G₂₂) occupied the 3′ sites for potential cleavage. Under equivalent conditions to those described above (FIG. 3), strand scission was observed uniquely at C₂₀ in OD4. Only low-level nonspecific reactivity was evident for OD3 (SEQ ID NO. 3) similar to that for OD1+OD2 (FIG. 3, lane 7). When the concentration of the compound of formula I was raised to 5 μM, nonspecific reactions also were evident for OD4 (SEQ ID NO. 4). Selective reaction at A₂₂ of OD1 and C₂₀ of OD4 and the absence of reaction at G₂₂ of OD3 indicated that recognition is not controlled by interaction with the target nucleobase. Truncation of the 5′ overhang of OD3 leaving a single nonhelical adenine led to loss of reaction at C₂₀ of OD4. In contrast, a more limited truncation of OD3 leaving a nonhelical 5′-GA continued to support reaction at C₂₀ of OD4. This reaction decreased to background levels when the 5′ guanine of the truncated OD3 was substituted with thymine, adenine, or cytosine. Therefore, the minimal requirement for recognition by a compound of formula I appeared to be a guanine at position n+1 on the 5′ overhang of OD3 (see FIG. 2). This result was consistent with that observed for OD1, wherein the n+1 position was also occupied by guanine (see FIG. 1).

### Example 4

### The synthesis of a trinuclear compound according to formula I or II is depicted in the Frey article on pages 330-331, which has been incorporated in its entirety.

1. A method of treating a cancer selected from the group consisting of leukemia, non-small cell lung cancer, colon cancer, central nervous system cancer, melanoma, ovarian cancer, renal cancer, ovarian cancer, cancer of the head and neck, bladder cancer, small cell cancer of the lung, squamous-cell carcinomas of the head, neck, esophagus, skin, and the genitourinary tract, including the cervix, vulva, scrotum, and penis, prostate cancer, and breast cancer, in a patient in need thereof, said method comprising administering to a patient a cancer-treating effective amount of a compound of formula I 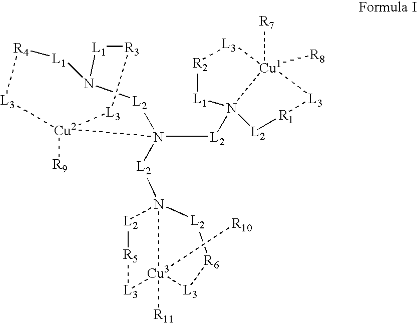
wherein
R₁-R₆ are each independently a 5 to 6 membered heterocycle containing 1-3 nitrogen atoms and optionally one oxygen atom, with the remainder of the atoms being carbon atoms, wherein the heterocycle is linked to a respective linkage L₃ through a nitrogen atom of the heterocycle, and wherein the heterocycle is linked to a respective linkage L₁ through any of the nitrogen or carbon atoms of the heterocycle other than the nitrogen atom that links to linkage L₃; and wherein the 5 to 6 membered heterocycle is unsubstituted or substituted with 1-3 substituents selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide;
R₇-R₁₁ are each independently an anion or uncharged species;
each L₁ is independently an ethyl, methyl, or ether linkage;
each L₃ is a direct bond; and
each L₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide, and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

2. A method of treating a cancer tumor, wherein the cancer tumor is selected from the group consisting of leukemia tumor, non-small cell lung tumor, colon cancer tumor, central nervous system cancer tumor, melanoma tumor, ovarian cancer tumor, renal cancer tumor, ovarian cancer tumor, cancer tumors of the head and neck, bladder cancer tumor, small cell cancer tumor of the lung, squamous-cell carcinoma tumors of the head, neck, esophagus, skin, and the genitourinary tract, including the cervix, vulva, scrotum, and penis, prostate cancer tumor, and breast cancer tumor, said method comprising administering to the cancer tumor a cancer tumor-treating effective amount of a compound of formula I 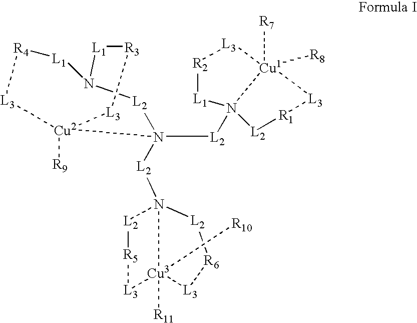
wherein
R₁-R₆ are each independently a 5 to 6 membered heterocycle containing 1-3 nitrogen atoms and optionally one oxygen atom, with the remainder of the atoms being carbon atoms, wherein the heterocycle is linked to a respective linkage L₃ through a nitrogen atom of the heterocycle, and wherein the heterocycle is linked to a respective linkage L₁ through any of the nitrogen or carbon atoms of the heterocycle other than the nitrogen atom that links to linkage L₃; and wherein the 5 to 6 membered heterocycle is unsubstituted or substituted with 1-3 substituents selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide;
R₇-R₁₁ are each independently an anion or uncharged species;
each L₁ is independently an ethyl, methyl, or ether linkage;
each L₃ is a direct bond; and
each L₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide, and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

3. A method of treating cancer cells, wherein the cancer cells selected from the group consisting of leukemia cells, non-small cell lung cancer cells, colon cancer cells, central nervous system cancer cells, melanoma cells, ovarian cancer cells, renal cancer cells, ovarian cancer cells, cancer cells of the head and neck, bladder cancer cells, small cell cancer cells of the lung, squamous-cell carcinoma cells of the head, neck, esophagus, skin, and the genitourinary tract, including the cervix, vulva, scrotum, and penis, prostate cancer cells, and breast cancer cells, said method comprising administering to the cancer cells a cancer cell-treating effective amount of a compound of formula I 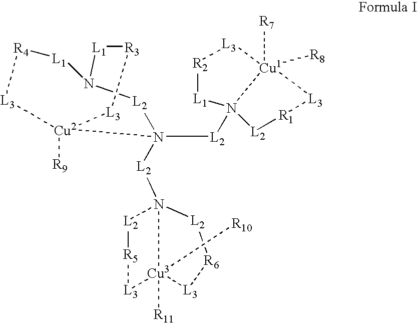
wherein
R₁-R₆ are each independently a 5 to 6 membered heterocycle containing 1-3 nitrogen atoms and optionally one oxygen atom, with the remainder of the atoms being carbon atoms, wherein the heterocycle is linked to a respective linkage L₃ through a nitrogen atom of the heterocycle, and wherein the heterocycle is linked to a respective linkage L₁ through any of the nitrogen or carbon atoms of the heterocycle other than the nitrogen atom that links to linkage L₃; and wherein the 5 to 6 membered heterocycle is unsubstituted or substituted with 1-3 substituents selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide;
R₇-R₁₁ are each independently an anion or uncharged species;
each L₁ is independently an ethyl, methyl, or ether linkage;
each L₃ is a direct bond; and
each L₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide, and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

4. (Cancelled)

5. (Cancelled)

6. A method of splitting a nucleic acid segment at a specific position thereon, wherein said method comprises
(a) providing a first nucleic acid segment having (i) an n position, wherein said n position is occupied by a first purine nucleotide that is non-complementary to a corresponding position x on a second nucleic acid segment, and (ii) an n+1 position which is occupied by a guanine residue, wherein said n+1 position is located directly adjacent to the n position upstream from the 5′ end of the first nucleic acid segment, and a second nucleic acid segment which is complementary to the first nucleic acid segment upstream from the position x, wherein the second nucleic acid segment is located either on a different or the same nucleic acid strand as the first nucleic acid segment;
(b) contacting at least the second nucleic acid segment with a compound of formula I for a time sufficient to split the nucleic acid at the position x of the second nucleic acid segment 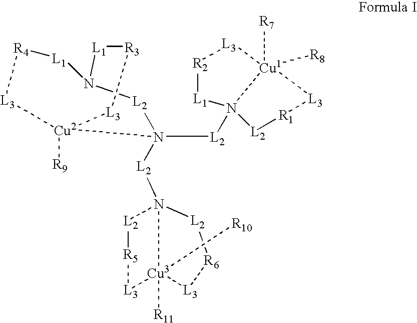
wherein
R₁-R₆ are each independently a 5 to 6 membered heterocycle containing 1-3 nitrogen atoms and optionally one oxygen atom, with the remainder of the atoms being carbon atoms, wherein the heterocycle is linked to a respective linkage L₃ through a nitrogen atom of the heterocycle, and wherein the heterocycle is linked to a respective linkage L₁ through any of the nitrogen or carbon atoms of the heterocycle other than the nitrogen atom that links to linkage L₃; and wherein the 5 to 6 membered heterocycle is unsubstituted or substituted with 1-3 substituents selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide;
R₇-R₁₁ are each independently an anion or uncharged species;
each L₁ is independently an ethyl, methyl, or ether linkage;
each L₃ is a direct bond; and
each L₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide, and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

7. (Cancelled)

8. A method of treating a cancer selected from the group consisting of leukemia, non-small cell lung cancer, colon cancer, central nervous system cancer, melanoma, ovarian cancer, renal cancer, ovarian cancer, cancer of the head and neck, bladder cancer, small cell cancer of the lung, squamous-cell carcinomas of the head, neck, esophagus, skin, and the genitourinary tract, including the cervix, vulva, scrotum, and penis, prostate cancer, and breast cancer, in a patient in need thereof, said method comprising administering to a patient a cancer-treating effective amount of a compound of formula II 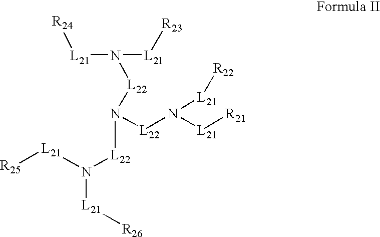
wherein
R₂₁-R₂₆ are each independently a 5 to 6 membered heterocycle containing 1-3 nitrogen atoms and optionally one oxygen atom, with the remainder of the atoms being carbon atoms, wherein the heterocycle is linked to a respective linker L₂₁ through a carbon or a nitrogen atom of the heterocycle; and wherein the 5 to 6 membered heterocycle is unsubstituted or substituted with halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, phenyloxycarbonylamino, naphthyloxycarbonylamino, semicarbazido, heteroaryl, 4-acetoxyphenyloxy, phenyl or acetamide;
each L₂₁ is independently an ethyl, methyl, or ether linkage; and
each L₂₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

9. A method of treating a cancer tumor, wherein the cancer tumor is selected from the group consisting of leukemia tumor, non-small cell lung cancer tumor, colon cancer tumor, central nervous system cancer tumor, melanoma tumor, ovarian cancer tumor, renal cancer tumor, ovarian cancer tumor, cancer tumors of the head and neck, bladder cancer tumor, small cell cancer tumor of the lung, squamous-cell carcinoma tumors of the head, neck, esophagus, skin, and the genitourinary tract, including the cervix, vulva, scrotum, and penis, prostate cancer tumor, and breast cancer tumor, said method comprising administering to the cancer tumor a cancer tumor-treating effective amount of a compound of formula II 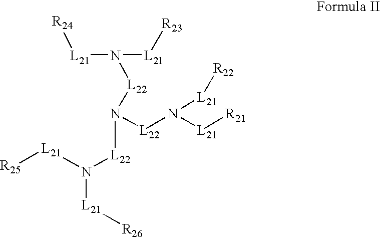
wherein
R₂₁-R₂₆ are each independently a 5 to 6 membered heterocycle containing 1-3 nitrogen atoms and optionally one oxygen atom, with the remainder of the atoms being carbon atoms, wherein the heterocycle is linked to a respective linker L₂₁ through a carbon or a nitrogen atom of the heterocycle; and wherein the 5 to 6 membered heterocycle is unsubstituted or substituted with halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, phenyloxycarbonylamino, naphthyloxycarbonylamino, semicarbazido, heteroaryl, 4-acetoxyphenyloxy, phenyl or acetamide;
each L₂₁ is independently an ethyl, methyl, or ether linkage; and
each L₂₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

10. A method of treating cancer cells, wherein the cancer cells selected from the group consisting of leukemia cells, non-small cell lung cancer cells, colon cancer cells, central nervous system cancer cells, melanoma cells, ovarian cancer cells, renal cancer cells, ovarian cancer cells, cancer cells of the head and neck, bladder cancer, small cell cancer cells of the lung, squamous-cell carcinoma cells of the head, neck, esophagus, skin, and the genitourinary tract, including the cervix, vulva, scrotum, and penis, prostate cancer cells, and breast cancer cells, said method comprising administering to the cancer cells a cancer cell-treating effective amount of a compound of formula II 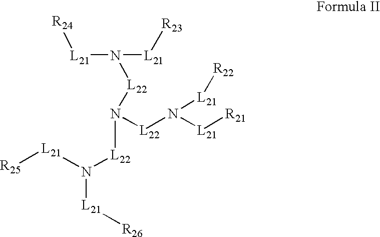
wherein
R₂₁-R₂₆ are each independently a 5 to 6 membered heterocycle containing 1-3 nitrogen atoms and optionally one oxygen atom, with the remainder of the atoms being carbon atoms, wherein the heterocycle is linked to a respective linker L₂₁ through a carbon or a nitrogen atom of the heterocycle; and wherein the 5 to 6 membered heterocycle is unsubstituted or substituted with halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, phenyloxycarbonylamino, naphthyloxycarbonylamino, semicarbazido, heteroaryl, 4-acetoxyphenyloxy, phenyl or acetamide;
each L₂₁ is independently an ethyl, methyl, or ether linkage; and
each L₂₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

11. (Cancelled)

12. (Cancelled)

13. The method of claim 6, wherein the complementary nucleic acid segment is located on the same nucleic acid strand as the nucleic acid segment.

14. The method of claim 6, wherein the complementary nucleic acid segment is located on a different nucleic acid strand as the nucleic acid segment.

15. A pharmaceutical composition containing a pharmaceutically effective amount of at least one compound of formula I 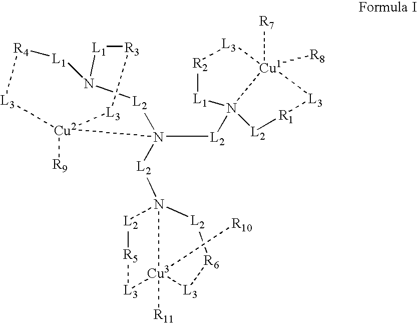
wherein
R₁-R₆ are each independently a 5 to 6 membered heterocycle containing 1-3 nitrogen atoms and optionally one oxygen atom, with the remainder of the atoms being carbon atoms, wherein the heterocycle is linked to a respective linkage L₃ through a nitrogen atom of the heterocycle, and wherein the heterocycle is linked to a respective linkage L₁ through any of the nitrogen or carbon atoms of the heterocycle other than the nitrogen atom that links to linkage L₃; and wherein the 5 to 6 membered heterocycle is unsubstituted or substituted with 1-3 substituents selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide;
R₇-R₁₁ are each independently an anion or uncharged species;
each L₁ is independently an ethyl, methyl, or ether linkage;
each L₃ is a direct bond; and
each L₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide, and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

16. A pharmaceutical composition containing a pharmaceutically effective amount of at least one compound of formula II 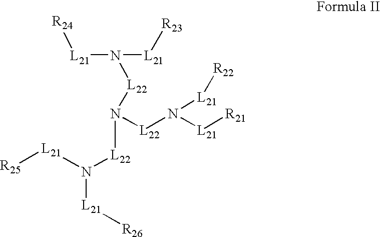
wherein
R₂₁-R₂₆ are each independently a 5 to 6 membered heterocycle containing 1-3 nitrogen atoms and optionally one oxygen atom, with the remainder of the atoms being carbon atoms, wherein the heterocycle is linked to a respective linker L₂₁ through a carbon or a nitrogen atom of the heterocycle; and wherein the 5 to 6 membered heterocycle is unsubstituted or substituted with halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, phenyloxycarbonylamino, naphthyloxycarbonylamino, semicarbazido, heteroaryl, 4-acetoxyphenyloxy, phenyl or acetamide;
each L₂₁ is independently an ethyl, methyl, or ether linkage; and
each L₂₂ is independently (a) a C₁-C₆ alkyl which may optionally be interrupted with one or more ether linkages, wherein the C₁-C₆ alkyl is unsubstituted or substituted with 1 to 3 substituents each independently selected from the group consisting of halogen, hydroxy, formyloxy, azido, carboxyl, cyano, amino, C₁-C₄ alkoxy, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; (b) an ether linkage; (c) an aromatic or cycloalkyl C₅-C₈ monocycle which is unsubstituted or substituted with 1 to 3 substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl and acetamide; and (d) an aromatic or cycloalkyl C₉-C₁₃ bicycle which is unsubstituted or substituted with 1 to 3 substituents, said substituents each independently selected from the group consisting of halogen, hydroxy, formyloxy, azido, carboxyl, cyano, amino, C₁-C₄ alkoxy, C₁-C₄ alkyl, benzyl, nitro, C₁-C₄ acylamino, formyl, formamido, thioformamido, C₁-C₄ alkoxycarbonylamino, C₁-C₄ alkoxycarbonyl, phenyloxycarbonylamino, naphthyloxycarbonylamino, semicarbazido, heteroaryl, 4-acetoxyphenyloxy, phenyl or acetamide.

17. The pharmaceutical composition of claim 15, wherein the pharmaceutical composition is pharmaceutically effective against leukemia, non-small cell lung cancer, colon cancer, central nervous system cancer, melanoma, ovarian cancer, renal cancer, ovarian cancer, cancer of the head and neck, bladder cancer, small cell cancer of the lung, squamous-cell carcinomas of the head, neck, esophagus, skin, and the genitourinary tract, including the cervix, vulva, scrotum, and penis, prostate cancer, and breast cancer.

18. The pharmaceutical composition of claim 16, wherein the pharmaceutical composition is pharmaceutically effective against leukemia, non-small cell lung cancer, colon cancer, central nervous system cancer, melanoma, ovarian cancer, renal cancer, ovarian cancer, cancer of the head and neck, bladder cancer, small cell cancer of the lung, squamous-cell carcinomas of the head, neck, esophagus, skin, and the genitourinary tract, including the cervix, vulva, scrotum, and penis, prostate cancer, and breast cancer.


SEQUENCE LISTING (4 sequences)

SEQ ID NO: 1 - DNA, 22 bp, Artificial Sequence
  Description of Artificial Sequence Synthetic oligonucleotide
tatgcctacagcatccagggtg

SEQ ID NO: 2 - DNA, 25 bp, Artificial Sequence
  Description of Artificial Sequence Synthetic oligonucleotide
caccctggatgctgtaggcataggc

SEQ ID NO: 3 - DNA, 26 bp, Artificial Sequence
  Description of Artificial Sequence Synthetic oligonucleotide
tgctcgaagtgccacctgacgtgatc

SEQ ID NO: 4 - DNA, 26 bp, Artificial Sequence
  Description of Artificial Sequence Synthetic oligonucleotide
cgtaacgtcaggtggcactctagcgt